(19) United States
(12) Patent Application Publication
(10) Pub. No.: US 2021/0228490 A1
(43) Pub. Date: Jul. 29, 2021

(54) STABLE PHARMACEUTICAL FORMULATION

(71) Applicant: ORYZON GENOMICS, S.A., Madrid (ES)

(72) Inventors: Reto MAURER, Barcelona (ES); Patrick BUSSON, Barcelona (ES); Georg HUMMEL, Barcelona (ES)

(73) Assignee: Oryzon Genomics, S.A., Madrid (ES)

(21) Appl. No.: 17/051,540

(22) PCT Filed: May 6, 2019
(86) PCT No.: PCT/EP2019/061576

§ 371 Date: Oct. 29, 2020

(30) Foreign Application Priority Data
May 4, 2018 (EP) 18170938.7

(51) Int. Cl. A61K 9/28; A61K 9/00; A61K 9/20

(52) U.S. Cl. CPC A61K 9/284; A61K 9/0053; A61K 9/2013; A61K 9/2018; A61K 9/2893; A61K 9/205; A61K 9/2031; A61K 9/2009; A61K 9/2054

(57) ABSTRACT

The present invention relates to a pharmaceutical composition of (trans)-N1-((1R,2S)-2-phenylcyclopropyl)cyclohexane-1,4-diamine, a process for the preparation thereof and its use in the treatment of diseases.

### FIELD OF THE INVENTION

The present invention relates to a pharmaceutical composition comprising (trans)-N1-((1R,2S)-2-phenylcyclopropyl)cyclohexane-1,4-diamine, a process for the preparation thereof and its use in the treatment of diseases.

### BACKGROUND OF THE INVENTION

A variety of chemical compounds have been reported for the treatment or prevention of a disease or condition in which LSD1(Lysine specific demethylase 1) inhibition plays a role or is implicated.

One such LSD1 inhibitor is (trans)-N1-((1R,2S)-2-phenylcyclopropyl)cyclohexane-1,4-diamine (CAS 1431304-21-0). This compound, and pharmaceutically acceptable salts thereof, in particular its dihydrochloride, has been described in WO 2013/057322, and its polymorphic forms as well as ways to its syntheses are described in WO 2016/177656.

(trans)-N1-((1R,2S)-2-phenylcyclopropyl)cyclohexane-1,4-diamine is useful in the treatment of diseases associated with LSD1, including cancer, in particular in the treatment of small-cell lung cancer and hematological cancers such as leukemia.

While (trans)-N1-((1R,2S)-2-phenylcyclopropyl)cyclohexane-1,4-diamine is chemically very stable as an API, it has been shown to decompose in presence of many excipients.

There is thus a need for a stable pharmaceutical formulation of (trans)-N1-((1R,2S)-2-phenylcyclopropyl)cyclohexane-1,4-diamine. The formulation should further be able to be produced in an easy and reproducible manner.

### SUMMARY OF THE INVENTION

The present invention relates to pharmaceutical compositions, in particular pharmaceutical compositions in solid dosage form for oral use, of (trans)-N1-((1R,2S)-2-phenylcyclopropyl)cyclohexane-1,4-diamine (i.e. the compound of Formula I depicted below) or pharmaceutically acceptable salts thereof, having improved stability. In particular, the inventors have surprisingly found that inclusion of a complexing agent, such as disodium edetate, and an antioxidant, such as ascorbic acid, results in pharmaceutical compositions having highly improved stability. Accordingly, the present invention relates to pharmaceutical compositions comprising (trans)-N1-((1R,2S)-2-phenylcyclopropyl)cyclohexane-1,4-diamine, or a pharmaceutically acceptable salt thereof, in particular its dihydrochloride, a complexing agent and an antioxidant.

### Definitions

The term “filler” refers to excipients that fill out the size of a tablet by increasing the bulk volume. Fillers make it possible for the final product to have the proper volume for patient handling. Examples of fillers include cellulose, lactose, starch, mannitol, etc. Specific examples are starch (like STA-RX 1500, CAS No. 9057-07-2), maize starch, mannitol (like Parteck® M100, Parteck® M200), isomalt (like GalenIQ™ 721) and microcrystalline cellulose (like Avicel® PH 101, Avicel® PH 102). Specific examples are mannitol and microcrystalline cellulose.

The term “binder” refers to excipients that hold the ingredients in a tablet together. Binders ensure that tablets and granules can be formed with required mechanical strength, and give volume to low active dose tablets. Examples of binders include polymers like polyvinlypyrrolidone (PVP, such as copovidone (PVP/VA 64), Povidone K30, etc.), hydroxypropyl methylcellulose (HPMC), hydroxypropylcellulose (HPC), maltodextrin (like Maltodextrin DE 15-18) and proteins like gelatin. A specific example is maltodextrin.

The term “lubricant” refers to excipients that prevent ingredients from clumping together and from sticking to the tablet punches or capsule filling machine. Lubricants also ensure that tablet formation and ejection can occur with low friction between active ingredient and wall. Examples of lubricants are minerals like talc or silica and fats like stearin, magnesium stearate, sodium stearyl fumarate, polyethylene glycol etc. A specific example is polyethylene glycol PEG 6000.

The term “complexing agent” or “complex former” refers to excipients which form complexes (particularly chelates) with alkaline earth and heavy-metal ions. The chelated form has few of the properties of the free ion, and for this reason chelating agents are often described as “removing” ions. Examples include disodium edetate (or other suitable forms of EDTA, for example, other edetate salts such as calcium disodium edetate) and gallium tetraazacyclododecane-1,4,7,10-tetraacetic acid. A specific example is disodium edetate.

The term “antioxidant” refers to excipients that inhibit the oxidation of other molecules and prevent the active pharmaceutical ingredient from oxidative decomposition. Examples include ascorbic acid and citric acid. A specific example is ascorbic acid.

The term “Film Coating System” refers to a system coating the kernel. Examples of film coating systems include Opadry®—based material and the like. The term “Opadry®—based material” refers to a “Film Coating System” like Opadry® II 31F265002 brown, Opadry® 32F265006 brown, Opadry® II 31K28690 white, Opadry® QX 321A265005 brown, Opadry® II 85F26792 brown, Opadry® II 85F18422 white, Opadry® II 85F205106 blue, Opadry® 85F220063 yellow etc.

The term “Coating Agent” refers to a material suitable as thin coat applied to a solid dosage form like a tablet. An example is polyvinyl alcohol.

The term “colorant” refers to a color changing agent like a white pigment. An example is titanium dioxide.

The term “plasticizer” refers to additives that decrease the plasticity or viscosity of a material. An example is Macrogol/PEG 3350.

The term “anti-caking agent” is a component in a coating system to prevent tackiness of the dosage forms during the manufacturing process. Examples are talc, glyceryl monostearate, magnesium stearate, silicon dioxide, and the like. A specific example is talc.

The ter “coating vehicle” or “processing liquid” refers to a material that helps adding the coating to the kernel. The coating vehicle is essentially removed during processing. Examples are organic solvents, water, and the like. A specific example is purified water.

A term like x±y % means the range from x %−y % to x %+y %. An example is 5±1%, which means the range from 4% (incl.) to 6% (incl.).

A term like “x±y %” or x % to y % in context with any filler, binder, complexing agent, antioxidant, lubricant and/or the compound of formula I or its pharmaceutically acceptable salts refers to “x±y %” or x % to y %, as the case may be, of the kernel's total weight. For example 10 mg of the compound of formula I in a tablet kernel of 200 mg is 5% of the compound of formula I. It will be understood that in the case of an uncoated solid dosage form (e.g., an uncoated tablet), any reference to the kernel relates to the entire uncoated solid dosage form.

A term like “x±y %” in context with any coating agent, colorant, plasticizer and/or anti-caking agent refers to “x±y %” of the film coating's total weight. For example 1.5 mg titanium dioxide in the tablet's coating of 6 mg is 25%.

The term “comprising the compound of formula I in a kernel” means that the compound of formula I is only in the kernel.

The corresponding pharmaceutically acceptable salts of the compound of formula I with acids can be obtained by standard methods known to the person skilled in the art, e.g. by dissolving the compound of formula I in a suitable solvent such as e.g. dioxane or tetrahydrofuran and adding an appropriate amount of the corresponding acid. The products can usually be isolated by filtration or by chromatography. Particular salts are hydrochloride, formate and trifluoroacetate. A specific example is hydrochloride salt, in particular dihydrochloride.

The term “pharmaceutically acceptable excipient” refers to carriers and auxiliary substances such as diluents, fillers, glidants, lubricants and the like that are compatible with the other ingredients of the formulation. Examples of corresponding non-toxic ingredients, which can be used for administration to human subjects, are well-known in the art.

The term “immediate release tablet” is a tablet or capsule formulated to release the active drug immediately after oral administration (a corresponding capsule may, e.g., have a gelatin shell encapsulating the kernel).

The term “disease associated with LSD1” refers to any disease or condition in which LSD1 plays a role, or where the disease or condition is associated with expression or activity of LSD1, or any disease or condition that can be treated or prevented by inhibiting LSD1 activity. Examples of diseases associated with LSD1 include cancer, beta-globinopathies (e.g. sickle cell disease), viral infections and other diseases linked to LSD1 activity.

Throughout the description and claims, unless indicated otherwise by explicit reference to a specific salt form of the compound of formula I, all amounts and % indicated in relation to a compound of formula I or a pharmaceutically acceptable salt thereof are expressed as amount or %, as the case may be, of the free base form.

### DETAILED DESCRIPTION OF THE INVENTION

The present invention relates to pharmaceutical compositions, in particular pharmaceutical compositions in solid dosage form for oral use, of (trans)-N1-((1R,2S)-2-phenylcyclopropyl)cyclohexane-1,4-diamine (i.e. the compound of Formula I depicted below) or pharmaceutically acceptable salts thereof, having improved stability. In particular, the inventors have surprisingly found that inclusion of a complexing agent, such as sodium edetate (particularly disodium edetate), and an antioxidant, such as ascorbic acid, results in pharmaceutical compositions having highly improved stability, as explained in more detail in Example 3. Accordingly, the present invention relates to pharmaceutical compositions comprising (trans)-N1-((1R,2S)-2-phenylcyclopropyl)cyclohexane-1,4-diamine, or a pharmaceutically acceptable salt thereof, in particular its dihydrochloride, a complexing agent and an antioxidant.

All embodiments of present invention can be combined, including any of the specific embodiments described further below.

The present invention relates to a pharmaceutical composition comprising a compound of formula I,

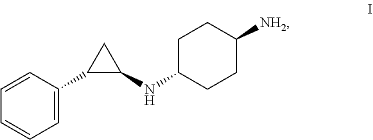

or a pharmaceutically acceptable salt thereof, a complexing agent and an antioxidant. This has the advantage to prevent decomposition of the compound of formula I.

E1: A specific embodiment of the present invention relates to a pharmaceutical composition comprising the dihydrochloride salt of the compound of formula I, a complexing agent and an antioxidant.

E2: A specific embodiment of the present invention relates to a pharmaceutical composition comprising the compound of formula I or a pharmaceutically acceptable salt thereof, a complexing agent, in particular (i.e. preferably) disodium edetate, and antioxidant, in particular (i.e. preferably) ascorbic acid or citric acid, more particularly (i.e. more preferably) ascorbic acid.

E3: A specific embodiment of the present invention relates to a pharmaceutical composition comprising the compound of formula I or a pharmaceutically acceptable salt thereof, a complexing agent, preferably disodium edetate, and an antioxidant selected from ascorbic acid and citric acid, preferably ascorbic acid.

E4: A specific embodiment of the present invention relates to a pharmaceutical composition comprising the dihydrochloride salt of the compound of formula I, a complexing agent, in particular disodium edetate, and an antioxidant selected from ascorbic acid and citric acid, more particularly ascorbic acid.

E5: A specific embodiment of the present invention relates to a pharmaceutical composition comprising the compound of formula I or a pharmaceutically acceptable salt thereof, disodium edetate and ascorbic acid.

E6: A specific embodiment of the present invention relates to a pharmaceutical composition comprising the dihydrochloride salt of the compound of formula I, disodium edetate and ascorbic acid.

E7: A specific embodiment of the present invention relates to the pharmaceutical composition as described herein, comprising 0.1% to 2%, in particular 0.2% to 1.6%, more particularly 0.3% to 0.5%, of a complexing agent, in particular disodium edetate.

E8: A specific embodiment of the present invention relates to the pharmaceutical composition as described herein, comprising 0.2% to 1.6%, in particular 0.3% to 0.5%, of a complexing agent, in particular disodium edetate.

E9: A specific embodiment of the present invention relates to the pharmaceutical composition as described herein, comprising 0.5%-10%, in particular 1% to 8%, more particularly 1.5% to 4%, most particularly 1.5% to 2.5%, of antioxidant, in particular ascorbic acid.

E10: A specific embodiment of the present invention relates to the pharmaceutical composition as described herein, comprising 1% to 8%, in particular 1.5% to 4%, more particularly 1.5% to 2.5%, of antioxidant, in particular ascorbic acid.

E11: A specific embodiment of the present invention relates to the pharmaceutical composition as described herein, comprising 0.1% to 2% of a complexing agent, and 0.5% to 10% of an antioxidant.

E12: A specific embodiment of the present invention relates to the pharmaceutical composition as described herein, comprising 0.1% to 2% of disodium edetate, and 0.5% to 10% of an antioxidant selected from ascorbic acid and citric acid, preferably ascorbic acid.

E13: A specific embodiment of the present invention relates to the pharmaceutical composition as described herein, comprising 0.2% to 1.6%, in particular 0.3% to 0.5%, of a complexing agent, and 1% to 8%, in particular 1.5% to 4%, more particularly 1.5% to 2.5%, of an antioxidant.

E14: A specific embodiment of the present invention relates to the pharmaceutical composition as described herein, comprising 0.2% to 1.6%, in particular 0.3% to 0.5%, of disodium edetate, and 1% to 8%, in particular 1.5% to 4%, more particularly 1.5% to 2.5%, of an antioxidant selected from ascorbic acid and citric acid, more particularly ascorbic acid.

E15: A specific embodiment of the present invention relates to the pharmaceutical composition as described herein in the form of a solid dosage form.

E16: A specific embodiment of the present invention relates to the pharmaceutical composition as described herein in the form of a solid dosage form for oral administration.

E17: A specific embodiment of the present invention relates to the pharmaceutical composition as described herein in the form of a solid dosage form comprising a kernel and a film coating system.

E18: A specific embodiment of the present invention relates to the pharmaceutical composition as described herein in the form of a solid dosage of or oral administration comprising a kernel and a film coating system.

E19: A specific embodiment of the present invention relates to the pharmaceutical composition as described herein in the form of a solid dosage form comprising a kernel and a coating.

E20: A specific embodiment of the present invention relates to the pharmaceutical composition as described herein in the form of a solid dosage form for oral administration comprising a kernel and a coating.

E21: A specific embodiment of the present invention relates to the pharmaceutical composition as described herein, comprising the compound of formula I or a pharmaceutically acceptable salt thereof in a kernel.

E22: A specific embodiment of the present invention relates to the pharmaceutical composition as described herein, comprising ≤1%, in particular ≤0.5% of the compound of formula I or a pharmaceutically acceptable salt thereof in a kernel, whereby the compound of formula I or a pharmaceutically acceptable salt thereof is present in said kernel.

E23: A specific embodiment of the present invention relates to the pharmaceutical composition as described herein, comprising 0.05% to 1%, in particular 0.05% to 0.5%, of the compound of formula I or a pharmaceutically acceptable salt thereof in a kernel.

E24: A specific embodiment of the present invention relates to the pharmaceutical composition as described herein, comprising the compound of formula I or a pharmaceutically acceptable salt thereof in a kernel, in particular 0.3%±0.25%, more particularly 0.2%±0.15%, more particularly 0.1% or 0.3%.

E25: A specific embodiment of the present invention relates to the pharmaceutical composition as described herein, comprising the compound of formula I or a pharmaceutically acceptable salt thereof in a kernel, in particular 0.2%±0.15%, more particularly 0.1% or 0.3%.

E26: A specific embodiment of the present invention relates to the pharmaceutical composition as described herein, comprising 0.2%±0.15%, of the compound of formula I or a pharmaceutically acceptable salt thereof in a kernel.

E27: A specific embodiment of the present invention relates to the pharmaceutical composition as described herein, comprising 0.1% or 0.3% of the compound of formula I or a pharmaceutically acceptable salt thereof in a kernel.

E28: A specific embodiment of the present invention relates to the pharmaceutical composition as described herein, comprising 0.1% of the compound of formula I or a pharmaceutically acceptable salt thereof in a kernel.

E29: A specific embodiment of the present invention relates to the pharmaceutical composition as described herein, comprising 0.3% of the compound of formula I or a pharmaceutically acceptable salt thereof in a kernel.

E30: A specific embodiment of the present invention relates to the pharmaceutical composition as described herein, comprising the dihydrochloride salt of the compound of formula I in a kernel.

E31: A specific embodiment of the present invention relates to the pharmaceutical composition as described herein, comprising ≤1%, in particular ≤0.5% of the dihydrochloride salt of the compound of formula I in a kernel, whereby the dihydrochloride salt of the compound of formula I is present in said kernel.

E32: A specific embodiment of the present invention relates to the pharmaceutical composition as described herein, comprising 0.05% to 1%, in particular 0.05% to 0.5%, of the dihydrochloride salt of the compound of formula I in a kernel.

E33: A specific embodiment of the present invention relates to the pharmaceutical composition as described herein, comprising the dihydrochloride salt of the compound of formula I in a kernel, in particular 0.3%±0.25%, in particular 0.25%±0.15%, more particularly 0.132% or 0.395%.

E34: A specific embodiment of the present invention relates to the pharmaceutical composition as described herein, comprising the dihydrochloride salt of the compound of formula I in a kernel, in particular 0.25%±0.15%, more particularly 0.132% or 0.395%.

E35: A specific embodiment of the present invention relates to the pharmaceutical composition as described herein, comprising 0.25%±0.15% of the dihydrochloride salt of the compound of formula I in a kernel.

E36: A specific embodiment of the present invention relates to the pharmaceutical composition as described herein, comprising 0.132% or 0.395% of the dihydrochloride salt of the compound of formula I in a kernel.

E37: A specific embodiment of the present invention relates to the pharmaceutical composition as described herein, comprising 0.132% of the dihydrochloride salt of the compound of formula I in a kernel.

E38: A specific embodiment of the present invention relates to the pharmaceutical composition as described herein, comprising 0.395% of the dihydrochloride salt of the compound of formula I in a kernel.

E39: A specific embodiment of the present invention relates to the pharmaceutical composition as described herein, further comprising at least one (or, preferably, all) of the following compounds in the kernel:
i) filler, in particular 85%±5% filler, ore particularly 85%±2% filler,
ii) binder, in particular 10%±3% binder, more particularly 10%±1% binder
iii) complexing agent, in particular 0.4%±0.3% complexing agent, more particularly 0.4%±0.1% complexing agent,
iv) antioxidant, in particular 2%±1% antioxidant, more particularly 2%±0.5% antioxidant, and
v) lubricant, in particular 2%±1% lubricant, more particularly 2%±0.5% lubricant.

E40: A specific embodiment of the present invention relates to the pharmaceutical composition as described herein, further comprising at least one (or, preferably, all) of the following compounds in the kernel:
i) filler, in particular 85%±2% filler, more particularly 85.205% or 85.468% filler,
ii) binder, in particular 10%±1% binder, more particularly 10%, binder
iii) complexing agent, in particular 0.4%±0.1% complexing agent, more particularly 0.4% complexing agent,
iv) antioxidant, in particular 2%±0.5% antioxidant, more particularly 2% antioxidant, and
v) lubricant, in particular 2%±0.5% lubricant, more particularly 2% lubricant.

E41: A specific embodiment of the present invention relates to the pharmaceutical composition as described herein, comprising the complexing agent and the antioxidant in a kernel, and further comprising at least one (or, preferably, all) of the following compounds in the kernel:
i) filler,
ii) binder, and
iii) lubricant.

E42: A specific embodiment of the present invention relates to the pharmaceutical composition as described herein, comprising the following compounds in the kernel:
i) filler, preferably 85%±5% filler, more preferably 85%±2% filler,
ii) binder, preferably 10%±3% binder, more preferably 10%±1% binder
iii) complexing agent, preferably 0.4%±0.3%, more preferably 0. %±0.1% complexing agent
iv) antioxidant, preferably 2%±1% antioxidant, more preferably 2%±0.5% antioxidant, and
v) lubricant, preferably 2%±1% lubricant, more preferably 2%±0.5% lubricant.

E43: A specific embodiment of the present invention relates to the pharmaceutical composition as described herein, further comprising at least one filler, in particular 85%±14% filler, more particularly 85%±10% filler, more particularly 85%±5% filler, more particularly 85%±2% filler, most particularly 85.205% or 85.468% filler in the kernel.

E44: A specific embodiment of the present invention relates to the pharmaceutical composition as described herein, further comprising at least one filler, in particular 85%±2% filler, more particularly 85.205% or 85.468% filler in the kernel.

E45: A specific embodiment of the present invention relates to the pharmaceutical composition as described herein, further comprising 85%±2% filler in the kernel.

E46: A specific embodiment of the present invention relates to the pharmaceutical composition as described herein, further comprising 85.205% or 85.468% filler in the kernel.

E47: A specific embodiment of the present invention relates to the pharmaceutical composition as described herein, further comprising 85.205% filler in the kernel.

E48: A specific embodiment of the present invention relates to the pharmaceutical composition as described herein, further comprising 85.468% filler in the kernel.

E49: A specific embodiment of the present invention relates to the pharmaceutical composition as described herein, further comprising at least one binder, in particular 10%±5% more particularly 10%±3% binder, more particularly 10%±1% binder, ore particularly 10% binder in the kernel.

E50: A specific embodiment of the present invention relates to the pharmaceutical composition as described herein, further comprising 10%±1% binder in the kernel.

E51: A specific embodiment of the present invention relates to the pharmaceutical composition as described herein, further comprising 10% binder in the kernel.

E52: A specific embodiment of the present invention relates to the pharmaceutical composition as described herein, comprising at least one complexing agent, in particular 0.4%±0.3% complexing agent, more particularly 0.4%±0.2% complexing agent, more particularly 0.4%±0.1%, most particularly 0.4% complexing agent in the kernel.

E53: A specific embodiment of the present invention relates to the pharmaceutical composition as described herein, comprising at least one complexing agent, in particular 0.4%±0.2% complexing agent, more particularly 0.4%±0.1% complexing agent, more particularly 0.4% complexing agent in the kernel.

E54: A specific embodiment of the present invention relates to the pharmaceutical composition as described herein, comprising at least one complexing agent, in particular 0.4%±0.1% complexing agent, more particularly 0.4% complexing agent in the kernel.

E55: A specific embodiment of the present invention relates to the pharmaceutical composition as described herein, comprising 0.4%±0.1% complexing agent in the kernel.

E56: A specific embodiment of the present invention relates to the pharmaceutical composition as described herein, comprising 0.4% complexing agent in the kernel.

E57: A specific embodiment of the present invention relates to the pharmaceutical composition as described herein, comprising at least one antioxidant, in particular 2%±1% antioxidant, more particularly 2%±0.8% antioxidant, more particularly 2%±0.5% antioxidant, more particularly 2% antioxidant in the kernel.

E58: A specific embodiment of the present invention relates to the pharmaceutical composition as described herein, comprising at least one antioxidant, in particular 2%±0.8% antioxidant, more particularly 2%±0.5% antioxidant, more particularly 2% antioxidant in the kernel.

E59: A specific embodiment of the present invention relates to the pharmaceutical composition as described herein, comprising at least one antioxidant, in particular 2%±0.5% antioxidant, more particularly 2% antioxidant in the kernel.

E60: A specific embodiment of the present invention relates to the pharmaceutical composition as described herein, comprising 2%±0.5% antioxidant in the kernel.

E61: A specific embodiment of the present invention relates to the pharmaceutical composition as described herein, comprising 2% antioxidant in the kernel.

E62: A specific embodiment of the present invention relates to the pharmaceutical composition as described herein, further comprising at least one lubricant, in particular 2%±1% lubricant, more particularly 2%±0.7%, more particularly 2%±0.5%, more particularly 2% lubricant in the kernel.

E63: A specific embodiment of the present invention relates to the pharmaceutical composition as described herein, further comprising at least one lubricant, in particular 2%±0.7% lubricant, ore particularly 2%±0.5%, more particularly 2% lubricant in the kernel.

E64: A specific embodiment of the present invention relates to the pharmaceutical composition as described herein, further comprising at least one lubricant, in particular 2%±0.5% lubricant, more particularly 2% lubricant in the kernel.

E65: A specific embodiment of the present invention relates to the pharmaceutical composition as described herein, further comprising 2%±0.5% lubricant in the kernel.

E66: A specific embodiment of the present invention relates to the pharmaceutical composition as described herein, further comprising 2% lubricant in the kernel.

E67: A specific embodiment of the present invention relates to the pharmaceutical composition as described herein, further comprising at least one (or, preferably, all) of the following compounds in the kernel:

i) 85%±2% filler,
ii) 10%±1% binder,
iii) 0.4%±0.1% complexing agent,
iv) 2%±0.5% antioxidant, and
v) 2%±0.5% lubricant.

E68: A specific embodiment of the present invention relates to the pharmaceutical composition as described herein, comprising the following compounds in the kernel:

i) 85%±2% filler,
ii) 10%±1% binder,
iii) 0.4%±0.1% complexing agent,
iv) 2%±0.5% antioxidant, and
v) 2%±0.5% lubricant.

E69: A specific embodiment of the present invention relates to the pharmaceutical composition as described herein, further comprising at least one (or, preferably, all) of the following compounds in the kernel:

i) 85.205% or 85.468% filler,
ii) 10% binder,
iii) 0.4% complexing agent,
iv) 2% antioxidant, and
v) 2% lubricant.

E70: A specific embodiment of the present invention relates to the pharmaceutical composition as described herein, further comprising at least one (or, preferably, all) of the following compounds in the kernel:

i) 85.205% filler,
ii) 10% binder,
iii) 0.4% complexing agent,
iv) 2% antioxidant, and
v) 2% lubricant.

E71: A specific embodiment of the present invention relates to the pharmaceutical composition as described herein, further comprising at least one (or, preferably, all) of the following compounds in the kernel:

i) 85.468% filler,
ii) 10% binder,
iii) 0.4% complexing agent,
iv) 2% antioxidant, and
v) 2% lubricant.

E72: A specific embodiment of the present invention relates to the pharmaceutical composition as described herein, wherein:
i) the fillers are mannitol and microcrystalline cellulose, preferably 64%±2% mannitol and 21%±1% microcrystalline cellulose,
ii) the binder is maltodextrin, preferably 10%±1% maltodextrin,
iii) the complexing agent is disodium edetate, preferably 0.4%±0.1 disodium edetate,
iv) the antioxidant is ascorbic acid, preferably 2%±0.5% ascorbic acid, and
v) the lubricant is polyethylene glycol 6000, preferably 2%±0.5% polyethylene glycol 6000.

E73: A specific embodiment of the present invention relates to the pharmaceutical composition as described herein, wherein
i) the fillers are mannitol and microcrystalline cellulose, in particular 64%±2% mannitol and 21%±1% microcrystalline cellulose, more particularly 64.098% mannitol and 21.370% microcrystalline cellulose or 63.965% mannitol and 21.240% microcrystalline cellulose,
ii) the binder is maltodextrin, in particular 10% maltodextrin,
iii) the complexing agent is disodium edetate, in particular 0.4% disodium edetate,
iv) the antioxidant is ascorbic acid, in particular 2% ascorbic acid, and
v) the lubricant is polyethylene glycol 6000, in particular 2% polyethylene glycol 6000.

E74: A specific embodiment of the present invention relates to the pharmaceutical composition as described herein, wherein

i) the fillers are 64%±2% mannitol and 21%±1% microcrystalline cellulose,
ii) the binder is 10% maltodextrin,
iii) the complexing agent is 0.4% disodium edetate,
iv) the antioxidant is 2% ascorbic acid, and
v) the lubricant is 2% polyethylene glycol 6000.

E75: A specific embodiment of the present invention relates to the pharmaceutical composition as described herein, wherein
i) the fillers are 64.098% mannitol and 21.370% microcrystalline cellulose or 63.965% mannitol and 21.240% microcrystalline cellulose,
ii) the binder is 10% maltodextrin,
iii) the complexing agent is 0.4% disodium edetate,
iv) the antioxidant is 2% ascorbic acid, and
v) the lubricant is 2% polyethylene glycol 6000.

E76: A specific embodiment of the present invention relates to the pharmaceutical composition as described herein, wherein

i) the fillers are 64.098% mannitol and 21.370% microcrystalline cellulose,
ii) the binder is 10% maltodextrin,
iii) the complexing agent is 0.4% disodium edetate,
iv) the antioxidant is 2% ascorbic acid, and
v) the lubricant is 2% polyethylene glycol 6000.

E77: A specific embodiment of the present invention relates to the pharmaceutical composition as described herein, wherein

i) the fillers are 63.965% mannitol and 2240% microcrystalline cellulose,
ii) the binder is 10% maltodextrin,
iii) the complexing agent is 0.4% disodium edetate,
iv) the antioxidant is 2% ascorbic acid, and
v) the lubricant is 2% polyethylene glycol 6000.

E78: A specific embodiment of the present invention relates to the pharmaceutical composition as described herein, further comprising a film coating system, in particular a film coating system comprising a coating agent (e.g., polyvinyl alcohol).

E79: A specific embodiment of the present invention relates to the pharmaceutical composition as described herein, further comprising a film coating system, in particular a film coating system comprising

i) a coating agent,
ii) a plasticizer,
iii) an anti-caking agent, and
iv) a colorant.

E80: A specific embodiment of the present invention relates to the pharmaceutical composition as described herein, further comprising a film coating system, in particular a film coating system comprising
i) a coating agent, in particular 2±0.5 mg/tablet coating agent, more particularly 2 mg/tablet coating agent,
ii) a plasticizer, in particular 1±0.25 mg/tablet plasticizer, more particularly 1.01 mg/tablet plasticizer,
iii) an anti-caking agent, in particular 0.75±0.25 mg/tablet anti-caking agent, more particularly 0.74 mg/tablet anti-caking agent, and
iv) a colorant, in particular 1.25±0.25 mg/tablet colorant, more particularly 1.25 mg/tablet colorant.

E81: A specific embodiment of the present invention relates to the pharmaceutical composition as described herein, further comprising a film coating system, in particular a film coating system comprising

i) 2±0.5 mg/tablet coating agent,
ii) 1±0.25 mg/tablet plasticizer,
iii) 0.75±0.25 mg/tablet anti-caking agent, and
iv) 1.25±0.25 mg/tablet colorant.

E82: A specific embodiment of the present invention relates to the pharmaceutical composition as described herein, further comprising a film coating system, in particular a film coating system comprising

i) 2 mg/tablet coating agent,
ii) 1.01 mg/tablet plasticizer,
iii) 0.74 mg/tablet anti-caking agent, and
iv) 1.25 mg/tablet colorant.

E83: A specific embodiment of the present invention relates to the pharmaceutical composition as described herein, further comprising a film coating system comprising a coating agent, in particular 2±0.5 m/tablet coating agent, more particularly 2 mg/tablet coating agent.

E84: A specific embodiment of the present invention relates to the pharmaceutical composition as described herein, further comprising a film coating system comprising a plasticizer, in particular 1±0.25 mg/tablet plasticizer, more particularly 1.01 mg/tablet plasticizer.

E85: A specific embodiment of the present invention relates to the pharmaceutical composition as described herein, further comprising a film coating system comprising an anti-caking agent, in particular 0.75±0.25 mg/tablet anti-caking agent, more particularly 0.74 mg/tablet anti-caking agent.

E86: A specific embodiment of the present invention relates to the pharmaceutical composition as described herein, further comprising a film coating system comprising a colorant, in particular 1.25±0.25 mg/tablet colorant, more particularly 1.25 mg/tablet colorant.

E87: A specific embodiment of the present invention relates to the pharmaceutical composition as described herein, wherein

i) the coating agent is polyvinyl alcohol,
ii) the plasticizer is polyethylene glycol 3350,
iii) the anti-caking agent is talc, and
iv) the colorant is titanium dioxide.

E88: A specific embodiment of the present invention relates to the pharmaceutical composition as described herein, wherein

i) the coating agent is polyvinyl alcohol, in particular 2 mg/tablet polyvinyl alcohol,
ii) the plasticizer is polyethylene glycol 3350, in particular 1.01 mg/tablet polyethylene glycol 3350,
iii) the anti-caking agent is talc, in particular 0.74 mg/tablet talc, and
iv) the colorant is titanium dioxide, in particular 1.25 mg/tablet titanium dioxide.

E89: A specific embodiment of the present invention relates to the pharmaceutical composition as described herein, wherein

i) the coating agent is 2 mg/tablet polyvinyl alcohol,
ii) the plasticizer is 1.01 mg/tablet polyethylene glycol 3350,
iii) the anti-caking agent is 0.74 mg/tablet talc, and
iv) the colorant is 1.25 mg/tablet titanium dioxide.

E90: A specific embodiment of the present invention relates to the pharmaceutical composition as described herein, wherein the kernel as described herein is film coated with 5% of a film coating system as described herein based on the kernel weight.

E91: A specific embodiment of the present invention relates to the pharmaceutical composition as described below

| | mg |
|---|---|
| Kernel | |
| a dihydrochloride salt of a compound | 0.132 |
| of formula I | |
| Mannitol | 64.098 |
| Microcrystalline Cellulose | 21.37 |
| Maltodextrin | 10 |
| Disodium Edetate | 0.4 |
| Ascorbic Acid | 2 |
| Polyethylene Glycol 6000 | 2 |
| Film Coating System | |
| Polyvinyl Alcohol | 2 |
| Polyethylene Glycol 3350 | 1.01 |
| Talc | 0.74 |
| Titanium Dioxide | 1.25 |

E92: A specific embodiment of the present invention relates to the pharmaceutical composition as described below

| | mg |
|---|---|
| Kernel | |
| a dihydrochloride salt of a compound | 0.395 |
| of formula I | |
| Mannitol | 63.965 |
| Microcrystalline Cellulose | 21.24 |
| Maltodextrin | 10 |
| Disodium Edetate | 0.4 |
| Ascorbic Acid | 2 |
| Polyethylene Glycol 6000 | 2 |
| Film Coating System | |
| Polyvinyl Alcohol | 2 |
| Polyethylene Glycol 3350 | 1.01 |
| Talc | 0.74 |
| Titanium Dioxide | 1.25 |

E93: A specific embodiment of the present invention relates to a process to produce the pharmaceutical composition as described herein, in particular a process comprising the steps described in FIG. 1.

E94: A specific embodiment of the present invention relates to a process to prepare a pharmaceutical composition according to the invention, comprising:
a) mixing a filler and optionally one or more portions of a binder to form a mixture,
b) wet granulating the mixture from step a) with a solution comprising the compound of formula I or a pharmaceutically acceptable salt thereof, a complexing agent, an antioxidant, one or ore portions of a binder and a solvent, followed by drying and optionally sieving the resulting granulate,
c) mixing the granulate obtained from step b) with a lubricant to form a mixture,
d) compressing the mixture obtained in step c) to form a tablet, and
e) optionally, coating the tablet with a film coating system. In some embodiments, the filler is mannitol and microcrystalline cellulose, the binder is maltodextrin, the complexing agent is disodium edetate, the antioxidant is ascorbic acid, the solvent is water (preferably purified water), and the lubricant is polyethylene glycol. In some embodiments, the tablet obtained in step d) is coated in step e) and the film coating system is Opadry II white. If a solid dosage form different from a tablet is prepared, steps d) and e) can be replaced by: d) processing the mixture obtained in step c) to form a solid dosage form.

E95: A specific embodiment of the present invention relates to the pharmaceutical composition as described herein for use as medicament.

E96: A specific embodiment of the present invention relates to the pharmaceutical composition as described herein for use in the treatment of a disease associated with LSD1.

E97: A specific embodiment of the present invention relates to the pharmaceutical composition as described herein for use in the treatment of cancer, such as, e.g., small-cell lung cancer, or a hematological cancer (more particularly leukemia, such as acute myeloid leukemia (AML)).

E98: A specific embodiment of the present invention relates to a method of treating a disease associated with LSD1, comprising administering to a patient in need thereof a pharmaceutical composition as described herein.

E99: A specific embodiment of the present invention relates to a method of treating cancer, such as small-cell lung cancer or a hematological cancer (e.g., leukemia, such as AML), comprising administering to a patient in need thereof a pharmaceutical composition as described herein.

E100: A specific embodiment of the present invention relates to the use of the pharmaceutical composition as described herein for the manufacture of a medicament for the treatment of cancer, such as small-cell lung cancer or a hematological cancer (e.g., leukemia, such as AML).

E101: A specific embodiment of the present invention relates to the immediate release tablet of a pharmaceutical composition as described herein.

E102: A specific embodiment of the present invention relates to a kit comprising the pharmaceutical composition as described herein and prescribing information also known as “leaflet”.

E103: A specific embodiment of the present invention relates to a tablet comprising a pharmaceutical composition as described herein.

E104: A specific embodiment of the present invention relates to a tablet consisting of a pharmaceutical composition as described herein.

E105: A specific embodiment of the present invention relates to a pharmaceutical composition as described herein for oral administration.

E106: A specific embodiment of the present invention relates to a pharmaceutical composition as described herein where the kernel is coated with an Opadry-coating system.

E107: A specific embodiment of the present invention relates to an immediate release tablet comprising the pharmaceutical composition as described herein.

E108: A specific embodiment of the present invention relates to an immediate release tablet consisting of the pharmaceutical composition as described herein.

E109: A specific embodiment of the present invention relates to an immediate release tablet as described herein that disintegrates within 3 minutes using water at 15-25° C.

E110: A specific embodiment of the present invention relates to a tablet as described herein with a dissolution ≥80% release of the compound of formula I using 0.1 NHCl at 15-25° C. within 30-35 minutes.

E111: A specific embodiment of the present invention relates to a process as described herein which uses fluid bed granulation of the kernel as described herein.

### BRIEF DESCRIPTION OF THE FIGURES

FIG. 1: A manufacturing process of a pharmaceutical composition as described herein.

FIG. 2: All tablet compositions without antioxidant and complex former (complexing agent) showed significant decrease of (trans)-N1-((1R,2S)-2-phenylcyclopropyl)cyclohexane-1,4-diamine content over time stored at 25° C./60% relative humidity. See Example 3 for more details.

FIGS. 3A and 3B: Granulates stored at 25° C./ambient humidity containing antioxidant and complex former exhibited highly improved stability compared to unprotected composition without antioxidant and complex former, which showed strong (trans)-N1-((1R,2S)-2-phenylcyclopropyl)cyclohexane-1,4-diamine decrease over time. FIG. 3A: composition 7 vs 8; FIG. 3B: composition 5 vs 6. See Example 3 for more details.

FIGS. 4A and 4B: Granulates stored at 50° C./ambient humidity containing antioxidant and complex former exhibited highly improved stability compared to unprotected composition without antioxidant and complex former, which showed strong (trans)-N1-((1R,2S)-2-phenylcyclopropyl)cyclohexane-1,4-diamine decrease over time. FIG. 4A: composition 7 vs 8; FIG. 4B: composition 5 vs 6. See Example 3 for more details.

FIG. 5: Tablets containing antioxidant and complex former described in examples 1 and 2 were stable at 25° C./60% relative humidity over a test period of 18 months and at 40° C./75% relative humidity over a test period of 6 months.

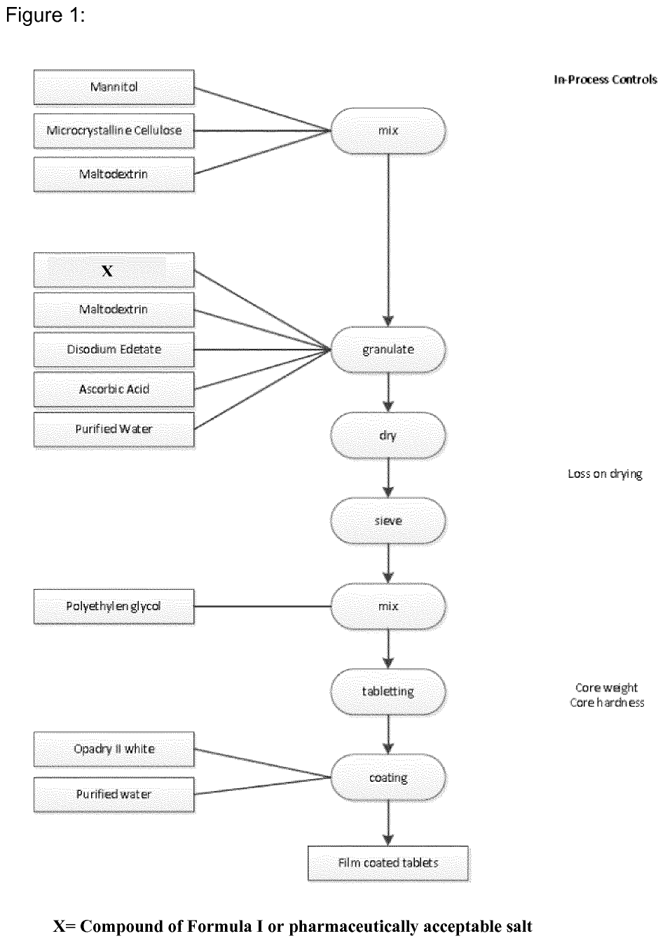

### EXPERIMENTAL PART

### Example 1: 100 μg Tablet

| | mg |
|---|---|
| Kernel | |
| a dihydrochloride salt of a compound | 0.132 |
| of formula I | |
| Mannitol | 64.098 |
| Microcrystalline Cellulose | 21.37 |
| Maltodextrin | 10 |
| Disodium Edetate | 0.4 |
| Ascorbic Acid | 2 |
| Polyethylene Glycol 6000 | 2 |
| Film Coating System | |
| Polyvinyl Alcohol | 2 |
| Polyethylene Glycol 3350 | 1.01 |
| Talc | 0.74 |
| Titanium Dioxide | 1.25 |

### Example 2: 300 μg Tablet

| | mg |
|---|---|
| Kernel | |
| a dihydrochloride salt of a compound | 0.132 |
| of formula I | |
| Mannitol | 63.965 |
| Microcrystalline Cellulose | 21.24 |
| Maltodextrin | 10 |
| Disodium Edetate | 0.4 |
| Ascorbic Acid | 2 |
| Polyethylene Glycol 6000 | 2 |
| Film Coating System | |
| Polyvinyl Alcohol | 2 |
| Polyethylene Glycol 3350 | 1.01 |
| Talc | 0.74 |
| Titanium Dioxide | 1.25 |

### Example 3: Stability

Excipient selection/composition development was conducted in order to stabilize (trans)-N1-((1R,2S)-2-phenylcyclopropyl)cyclohexane-1,4-diamine in the final drug product to prevent its chemical degradation. (trans)-N1-((1R,2S)-2-phenylcyclopropyl)cyclohexane-1,4-diamine is very unstable in presence of excipients which are used to form a tablet composition. This is reflected by a decrease of the (trans)-N1-((1R,2S)-2-phenylcyclopropyl)cyclohexane-1,4-diamine content (assay) over time upon storage. The effect is more pronounced at elevated storage temperature.

Tablets containing 0.1 and 0.3% (trans)-N1-((1R,2S)-2-phenylcyclopropyl)cyclohexane-1,4-diamine respectively were developed and stability assessment performed. The other excipients were Mannitol (filler), maize starch (filler), microcrystalline cellulose (filler), corn starch (filler), Maltodextrin or Hydroxypropyl methylcellulose (binder), crospovidone (disintegrant), sodium stearylfumarate (glidant) and commercially available Opadry® white (coating agent). Their composition is indicated in the table below:

| | Comp. 1 | Comp. 2 | Comp. 3 | Comp. 4 |
|---|---|---|---|---|
| | mg | mg | mg | mg |
| Kernel | | | | |
| a dihydrochloride salt of a | 0.132 | 0.396 | 0.132 | 0.132 |
| comound of formula I | | | | |
| Mannitol | 27.87 | 27.60 | 38.74 | 39.5 |
| Microcrystalline Cellulose | 25.00 | 25.00 | 15.00 | 15.00 |
| Maltodextrin | | | | 4.34 |
| Starch 1500 | 15.00 | 15.00 | 15.00 | 15.00 |
| Corn Starch | 20.00 | 20.00 | 20.00 | 20.00 |
| Copovidone (Kollidon VA | | | 5.13 | |
| 64) | | | | |
| Crospovidone | 4.00 | 4.00 | 4.00 | 4.00 |
| Sodium Stearyl Fumarate | 2.00 | 2.00 | 2.00 | 2.00 |
| Hydroxypropyl | 6.00 | 6.00 | | |
| methylcelullose | | | | |
| Film Coating System | | | | |
| Polyvinyl Alcohol | 2 | 2 | 2 | 2 |
| Polyethylene Glycol 3350 | 1.01 | 1.01 | 1.01 | 1.01 |
| Talc | 0.74 | 0.74 | 0.74 | 0.74 |
| Titanium Dioxide | 1.25 | 1.25 | 1.25 | 1.25 |

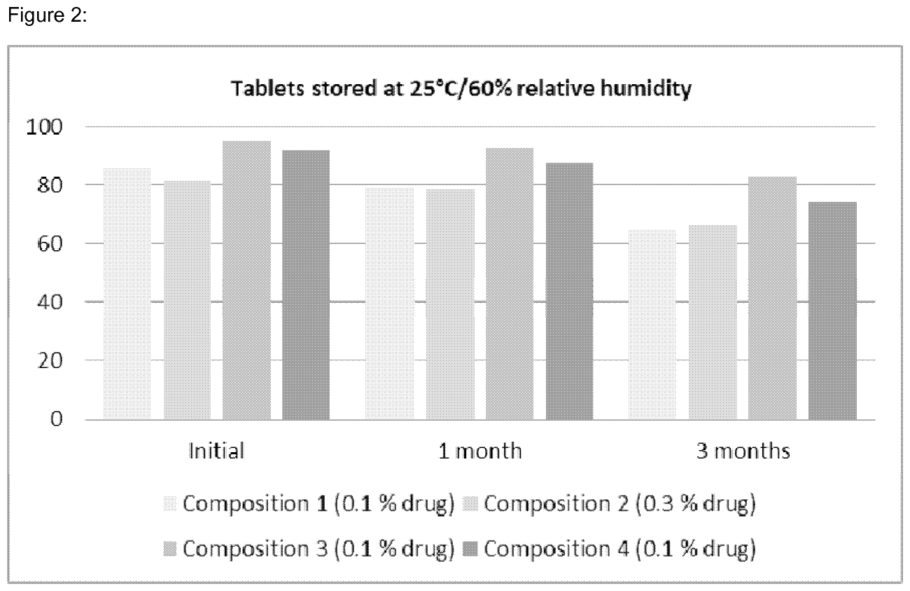

All tablet compositions showed significant decrease of (trans)-N1-((1R,2S)-2-phenylcyclopropyl)cyclohexane-1,4-diamine content overtime stored at 25° C./60% relative humidity (FIG. 2).

Decrease of (trans)-N1-(1R,2S)-2-phenylcyclopropyl)cyclohexane-1,4-diamine content was potentially caused by metal-ion induced oxidative degradation. Therefore metal-complexing excipients as well as antioxidants were introduced to the composition that prevent degradation. These components are here not part of liquid or semi-solid compositions. A number of granulates with different compositions were manufactured with and without antioxidant (ascorbic acid) and complexing agent (disodium edetate) and stability assessment was performed Their composition is indicated in the table below:

| | Comp. 5 | Comp. 6 | Comp. 7 | Comp. 8 |
|---|---|---|---|---|
| Granulate | mg | mg | mg | mg |
| a dihydrochloride salt of a | 0.396 | 0.396 | 0.396 | 0.396 |
| compound of formula I | | | | |
| Isomalt GalenIQ801 | 72.28 | 70.36 | | |
| MCC Vivapur 101 | 17.32 | 16.84 | | |
| Croscarmellose Sodium | 3.00 | 3.00 | | |
| Aerosi1200 | 2.00 | 2.00 | | |
| Mannitol | | | 27.60 | 25.07 |
| Microcrystalline Cellulose | | | 25.0 | 25.0 |
| Starch 1500 | | | 15.00 | 15.00 |
| Corn Starch | | | 20.00 | 20.00 |
| HPMC 2910 3cp | | | 6.00 | 6.00 |
| Disodium Edetate | | 0.4 | | 0.42 |
| Ascorbic Acid | | 2.00 | | 2.11 |

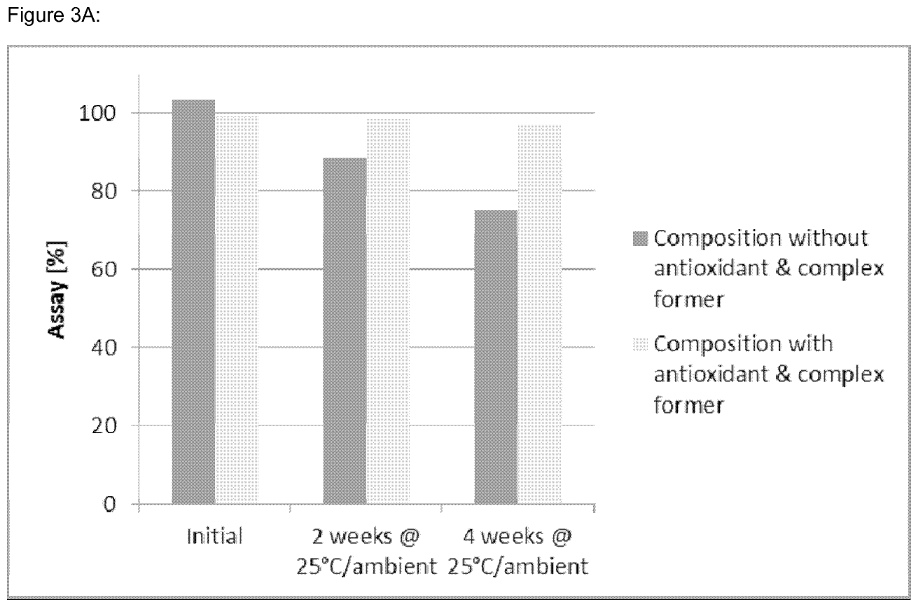

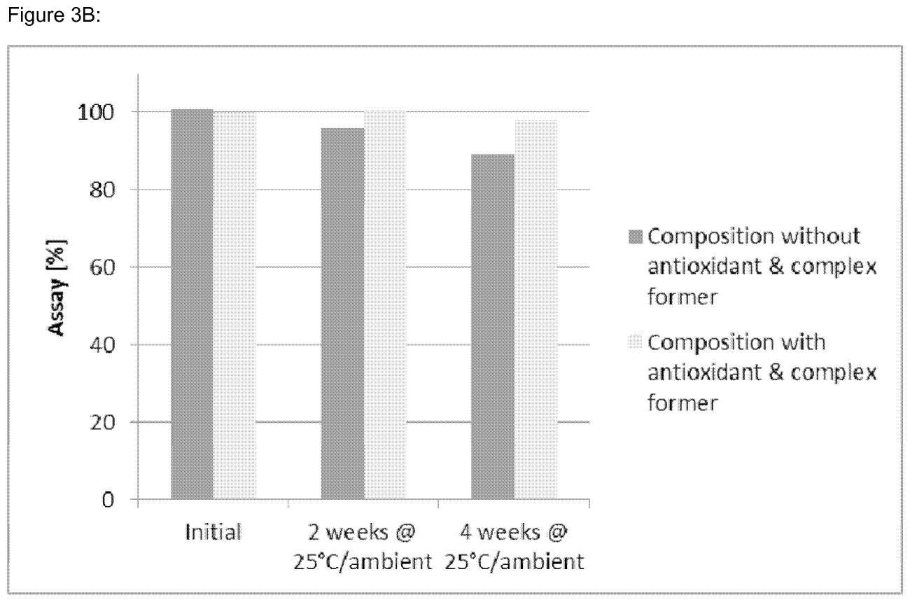

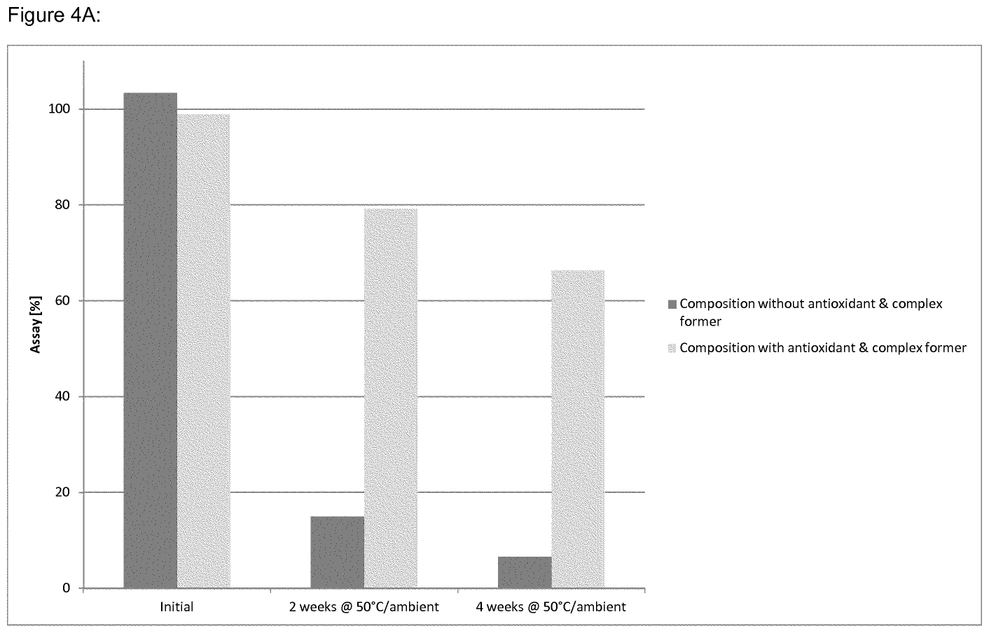

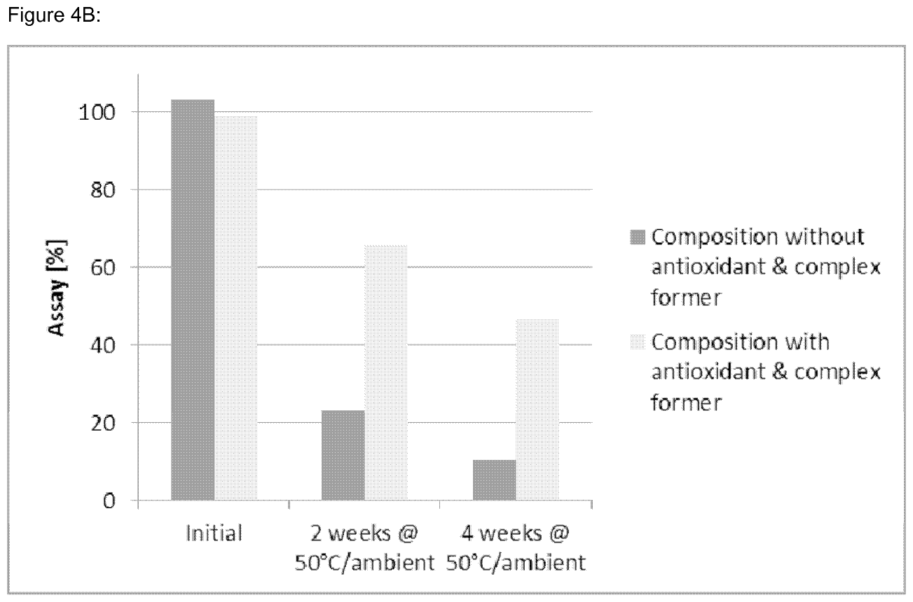

Granulates stored at 25° C./60% relative humidity containing ascorbic acid as antioxidant and disodium edetate as complexing agent (compositions 6 and 8) exhibited good stability, whereas unprotected compositions (compositions 5 and 7) showed strong (trans)-N1-((1R,2S)-2-phenylcyclopropyl)cyclohexane-1,4-diamine decrease over time (FIG. 3A: comparison of stability of composition 7 vs 8, and FIG. 3B: comparison of stability of composition 5 vs 6). The effect was even more pronounced at elevated temperature (50° C.) (FIG. 4A: composition 7 vs 8; FIG. 4B: composition 5 vs 6).

As shown by the data in FIGS. 3 and 4, the combination of antioxidant and complexing agent clearly improved the API stability independently from the underlying formulation composition.

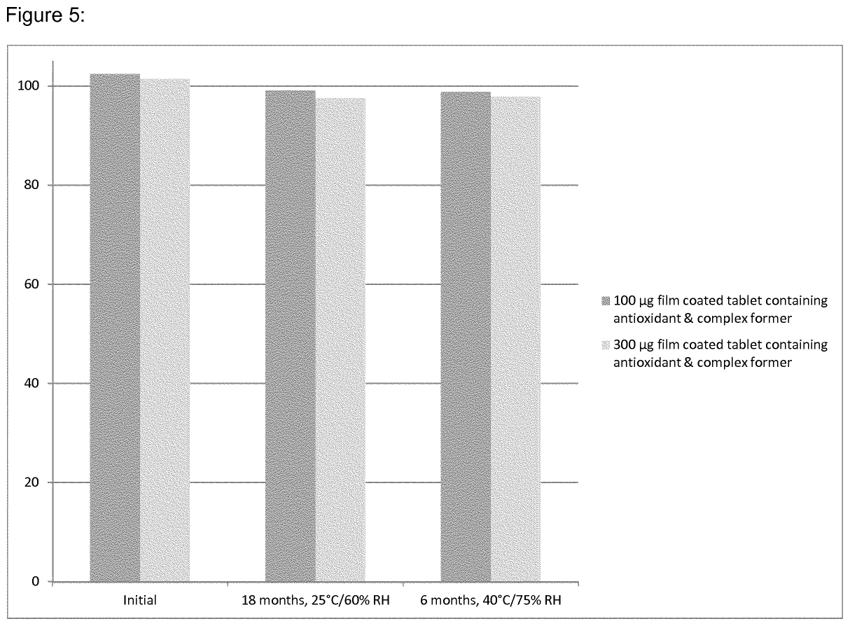

The benefit of antioxidant and complexing agent is clearly shown and therefore applied for examples 1 and 2, which were prepared using ascorbic acid as antioxidant and disodium edetate as complexing agent. Stability was confirmed and assay within specification over a test period of 18 months at every test storage condition. As a way of example, results of stability testing for the tablets of Examples 1 and 2 at 25° C./60% relative humidity over a test period of 18 months and at 40° C./75% relative humidity over a test period of 6 months are provided in FIG. 5, showing these tablets containing antioxidant (ascorbic acid) and complexing agent (disodium edetate) are stable.

### Example 4: Process

Examples 1 and 2 are manufactured using a fluid bed granulation process. Standard fluid bed granulation is performed with a powder blend containing API and excipients which is granulated with a binder solution.

As target drug load for (trans)-N1-((1R,2S)-2-phenylcyclopropyl)cyclohexane-1,4-diamine was only 0.1 (example 1) and 0.3% (example 2) respectively, the process was adapted in order to achieve desired drug uniformity in the final drug product. (trans)-N1-((1R,2S)-2-phenylcyclopropyl)cyclohexane-1,4-diamine, ascorbic acid and disodium edetate were dissolved in the maltodextrin solution and carefully sprayed upon the moving powder bed containing mannitol and microcrystalline cellulose. Content uniformity was achieved.

Alternatively, in order to prevent the ascorbic acid causing precipitation of disodium edetate due to pH-shift, the granulation liquid was divided into two phases; one containing maltodextrin and Na₂-EDTA and the other maltodextrin and ascorbic acid. The two solutions were fed separately to the fluid bed drier and united via Y-fitting after passing the peristaltic pump shortly before entering the bi-fluid nozzle.

Stability results of batches with the composition of examples 1 and 2 manufactured with two separated granulation liquids and single granulation liquid exhibited assay within specification over a test period of 12 months at 25° C./60% relative humidity and over a test period of 6 months at 40° C./75% relative humidity.

1. A pharmaceutical composition comprising a compound of formula I, 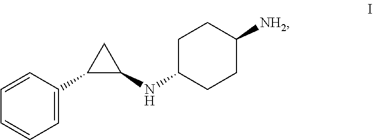 [SMILES: N[C@H]1CC[C@H](N[C@@H]2C[C@H]2c2ccccc2)CC1]
or a pharmaceutically acceptable salt thereof, a complexing agent and an antioxidant.

2. The pharmaceutical composition according to claim 1, comprising the dihydrochloride salt of the compound of formula I, a complexing agent and an antioxidant.

3. The pharmaceutical composition according to any one of claim 1 or 2, wherein the complexing agent is disodium edetate, and the antioxidant is ascorbic acid.

4. The pharmaceutical composition according to any one of claims 1-3, wherein the pharmaceutical composition is a solid dosage form for oral administration.

5. The pharmaceutical composition according to any one of claims 1-4, wherein the pharmaceutical composition is a solid dosage form comprising a kernel and a film coating system.

6. The pharmaceutical composition according to any one of claims 1-5, comprising the compound of formula I or a pharmaceutically acceptable salt thereof in a kernel.

7. The pharmaceutical composition according to any one of claims 1-6, comprising the dihydrochloride salt of the compound of formula I in a kernel.

8. The pharmaceutical composition according to any one of claims 1-7, comprising the complexing agent and the antioxidant in a kernel, and further comprising at least one of the following compounds in the kernel:
i) filler,
ii) binder, and
iii) lubricant.

9. The pharmaceutical composition according to claim 7 or 8, comprising the following compounds in the kernel:
i) filler, preferably 85%±2% filler,
ii) binder, preferably 10%±1% binder,
iii) complexing agent, preferably 0.4%±0.1% complexing agent,
iv) antioxidant, preferably 2%±0.5% antioxidant, and
v) lubricant, preferably 2% t 0.5% lubricant.

10. The pharmaceutical composition according to claim 9, wherein
i) the fillers are mannitol and microcrystalline cellulose, preferably 64%±2% mannitol and 21%±1% microcrystalline cellulose,
ii) the binder is maltodextrin, preferably 10%±1% maltodextrin,
iii) the complexing agent is disodium edetate, preferably 0.4%±0.1% disodium edetate,
iv) the antioxidant is ascorbic acid, preferably 2%±0.5% ascorbic acid, and
v) the lubricant is polyethylene glycol 6000, preferably 2%±0.5% polyethylene glycol 6000.

11. The pharmaceutical composition according to any one of claims 1-10, comprising a film coating system, preferably a film coating system comprising:
i) a coating agent,
ii) a plasticizer,
iii) an anti-caking agent, and
iv) a colorant.

12. The pharmaceutical composition according to any one of claims 1-11, comprising a film coating system comprising:
i) 2±0.5 mg/tablet coating agent,
ii) 1±0.25 mg/tablet plasticizer,
iii) 0.75±0.25 mg/tablet anti-caking agent, and
iv) 1.25±0.25 mg/tablet colorant.

13. The pharmaceutical composition according to claim 11 or 12, wherein
i) the coating agent is polyvinyl alcohol,
ii) the plasticizer is polyethylene glycol 3350,
iii) the anti-caking agent is tale, and
iv) the colorant is titanium dioxide.

14. The pharmaceutical composition according to any one of claims 1-13, wherein the kernel according to any one of claims 5-10 is film coated with 5% of a film coating system according to any one of claims 11-13 based on the kernel weight.

15. A process to produce the pharmaceutical composition as described in any one of claims 1-14, comprising:
a) mixing a filler and optionally one or more portions of a binder to form a mixture,
b) wet granulating the mixture from step a) with a solution comprising the compound of formula I or a pharmaceutically acceptable salt thereof, a complexing agent, an antioxidant, one or more portions of a binder and a solvent, followed by drying and optionally sieving the resulting granulate,
c) mixing the granulate obtained from step b) with a lubricant to form a mixture,
d) compressing the mixture obtained in step c) to form a tablet, and
e) optionally, coating the tablet with a film coating system.

16. The pharmaceutical composition as described in any one of claims 1-14 for use as medicament.

17. The pharmaceutical composition as described in any one of claims 1-14 for use in the treatment of a disease associated with LSD1.

18. The pharmaceutical composition as described in any one of claims 1-14 for use in the treatment of cancer.

19. A method of treating a disease associated with LSD1, comprising administering to a patient in need thereof a pharmaceutical composition as described in any one of claims 1-14.

20. A method of treating cancer, comprising administering to a patient in need thereof a pharmaceutical composition as described in any one of claims 1-14.

21. The pharmaceutical composition for use of claim 18 or the method of claim 20, wherein the cancer is small cell lung cancer or leukemia.

22. An immediate release tablet comprising a pharmaceutical composition as described in any one of claims 1-14.
